(19) United States
(12) Patent Application Publication
(10) Pub. No.: US 2021/0290581 A1
(43) Pub. Date: Sep. 23, 2021

(54) NEUROREGENERATION IMPROVED BY KETONE

(71) Applicant: UNIVERSITY OF SOUTH FLORIDA, Tampa, FL (US)

(72) Inventors: Csilla ARI D'AGOSTINO, Tampa, FL (US); Dominic Paul D'AGOSTINO, Tampa, FL (US)

(21) Appl. No.: 16/332,594

(22) PCT Filed: Sep. 12, 2017
(86) PCT No.: PCT/US2017/051125

§ 371 Date: Mar. 12, 2019

Related U.S. Application Data
(60) Provisional application No. 62/393,233, filed on Sep. 12, 2016.

(51) Int. Cl. A61K 31/225; A61P 25/28

(52) U.S. Cl. CPC A61K 31/225; A61P 25/28

(57) ABSTRACT

Disclosed herein are compositions and methods for treating nervous system injury in a subject.

### CROSS-REFERENCE TO RELATED APPLICATIONS

This application claims priority to U.S. Provisional Application No. 62/393,233, filed on Sep. 12, 2016, which is incorporated fully herein by reference.

### TECHNICAL FIELD

This invention relates to methods for treating nervous system injury. Specifically, the invention describes methods of treating nervous system injury using ketogenic compositions.

### BACKGROUND

Nervous system injuries affect a significant number of people every year and result in damage to nervous tissue, which in turn can cause impairment and/or loss of certain motor functions, brain functions, etc. When the nervous tissue is damaged, along with the underlying neurons, glia, axons, myelin, and synapses, the availability of glucose is limited for fueling brain function. As such, nervous tissue must undergo regrowth or repair in order to restore proper functionality.

There are two types of nervous system injury: injury to the peripheral nervous system (PNS) and injury to the central nervous system (CNS). The peripheral nervous system has an intrinsic ability to repair itself, but the central nervous system is incapable of making such a repair. Currently, there is no known method of repairing and regenerating central nervous system tissue and its underlying components. Accordingly, what is needed is a method of repairing and regenerating of neuronal cells after CNS injury in order to improve patient outcome.

### SUMMARY

Disclosed herein are methods for treating nervous system injury in a subject. The nervous system injury may be a CNS injury. The CNS injury may be a brain or a spinal cord injury. The nervous system injury may be a peripheral nervous system injury. The method may comprise administering a ketogenic composition to the subject. The ketogenic composition may comprise one or more ketogenic compounds selected from the group consisting of a ketone ester, a ketone salt, a ketone body precursor, and a combination thereof. The ketone ester may be 1,3-butanediol-acetoacetate diester. The ketone salt may be R,S-sodium-3-hydroxybutyrate. The composition may enhance neural regeneration following nervous system injury.

### BRIEF DESCRIPTION OF THE DRAWINGS

FIG. 1A depicts a scratch assay of the control group at time 0 hours.

FIG. 1B depicts a scratch assay of the control group at time 24 hours.

FIG. 2A is depicts a scratch assay of the treated group at time 0 hours.

FIG. 2B depicts a scratch assay of the control group at time 24 hours.

FIG. 3 depicts a scratch assay at 24 hours showing DAPI nucleus stain in the control group versus the treated group.

FIG. 4 is a graphical illustration comparing the treated group and the control group, particularly with regards to the number of nuclei in the scratch space area after 24 hours at 10× and 20× magnification.

FIG. 5 is a graphical illustration comparing the treated group and the control group, particularly with regards to increase in cell coverage per 24 hours.

FIG. 6A depicts fluorescence imaging of cell nuclei, Beta tubulin, and synapsins in the control group at 10×.

FIG. 6B depicts fluorescence imaging of cell nuclei, Beta tubulin, and synapsins in the treated group at 10×.

FIG. 7A depicts fluorescence imaging of cell nuclei, Beta tubulin, and synapsins in the control group at 20×.

FIG. 7B depicts fluorescence imaging of cell nuclei, Beta tubulin, and synapsins in the treated group at 20×.

FIG. 8A depicts fluorescence imaging of cell nuclei, Beta tubulin, and synapsins in the control group at 20×.

FIG. 8B depicts fluorescence imaging of cell nuclei, Beta tubulin, and synapsins in the treated group at 20×.

FIG. 9A depicts fluorescence imaging of cell nuclei, Beta tubulin, and synapsins in the control group at 60×.

FIG. 9B depicts fluorescence imaging of cell nuclei, Beta tubulin, and synapsins in the treated group at 60×.

### DETAILED DESCRIPTION

The present disclosure describes a method of treating nervous system injury in a subject, comprising administering to the subject a composition comprising one or more ketogenic compounds.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

The term “administration” or “administering” is used throughout the specification to describe the process by which the disclosed ketogenic compositions may be delivered to a subject. Administration will often depend upon the amount of composition administered, the number of doses, and duration of treatment. Multiple doses of the composition may be administered. The frequency and duration of administration of the composition can vary, depending on any of a variety of factors, including patient response, etc. The ketogenic compositions may be administered to the subject by any suitable route. The compositions may be administered orally, parenterally, (including intravenous, subcutaneous, topical, transdermal, intradermal, transmucosal, intraperitoneal, intramuscular, intracapsular, intraorbital, intracardiac, transtracheal, subcutaneous, subcuticular, intraarticular, subcapsular, subarachnoid, intraspinal, and epidural injection) by infusion, by electroporation, or co-administered as a component of any medical device or object to be inserted (temporarily or permanently) into a subject.

The amount of the composition administered can vary according to factors such as the degree of susceptibility of the individual, the age, sex, and weight of the individual, idiosyncratic responses of the individual, the dosimetry, and the like. Detectably effective amounts of the ketogenic composition can also vary according to instrument and film-related factors. Optimization of such factors is well within the level of skill in the art, unless otherwise noted.

The term “aliphatic group” is defined as including alkyl, alkenyl, alkynyl, halogenated alkyl and cycloalkyl groups as defined above. A “lower aliphatic group” is an aliphatic group that contains from 1 to 10 carbon atoms.

The term “alkoxy group” is represented by the formula —OR, where R can be an alkyl group, including a lower alkyl group, optionally substituted with an alkenyl, alkynyl, aryl, aralkyl, cycloalkyl, halogenated alkyl, or heterocycloalkyl group, as defined below.

The term “alkenyl group” is defined as a hydrocarbon group of 2 to 24 carbon atoms and structural formula containing at least one carbon-carbon double bond.

The term “alkyl group” is defined as a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. A “lower alkyl” group is a saturated branched or unbranched hydrocarbon having from 1 to 10 carbon atoms.

The term “alkynyl group” is defined as a hydrocarbon group of 2 to 24 carbon atoms and a structural formula containing at least one carbon-carbon triple bond.

The term “aralkyl” is defined as an aryl group having an alkyl group, as defined above, attached to the aryl group. An example of an aralkyl group is a benzyl group.

The term “aryl group” is defined as any carbon-based aromatic group including, but not limited to, benzene, naphthalene, etc. The term “aromatic” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorous. The aryl group can be substituted with one or more groups including, but not limited to, alkyl, alkynyl, alkenyl, aryl, halide, nitro, amino, ester, ketone, aldehyde, hydroxy, carboxylic acid, or alkoxy, or the aryl group can be unsubstituted.

As used herein “beta-hydroxybutyrate,”, “βHB”, or “BHB” as used interchangeably herein refer to a carboxylic acid having the general formula CH₃CH₂OHCH₂COOH. BHB is a ketone body which may be utilized by the body as a fuel source during instances of low glucose levels.

The terms “CNS injury” or “central nervous system injury” as used interchangeably herein refers to any injury to the central nervous system. CNS injury may be a brain injury. A brain injury may be any injury to the brain. CNS injury may be a spinal cord injury (SCI). Spinal cord injury may be any injury to the spinal cord.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

The term “cycloalkyl group” is defined as a non-aromatic carbon-based ring composed of at least three carbon atoms. Examples of cycloalkyl groups include, but are not limited to, cyclopropyl, cyclobutyl, cyclopentyl, cyclohexyl, etc.

“Derivative” refers to a compound or portion of a compound that is derived from or is theoretically derivable from a parent compound.

The term “ester” as used herein is represented by the formula —OC(O)R, where R can be an alkyl, alkenyl, alkynyl, aryl, aralkyl, cycloalkyl, halogenated alkyl, or heterocycloalkyl group.

“Esterification” refers to the reaction of an alcohol with a carboxylic acid or a carboxylic acid derivative to give an ester.

The term “halogenated alkyl group” is defined as an alkyl group as defined above with one or more hydrogen atoms present on these groups substituted with a halogen (F, Cl, Br, I).

The term “heterocycloalkyl group” is a cycloalkyl group as defined above where at least one of the carbon atoms of the ring is substituted with a heteroatom such as, but not limited to, nitrogen, oxygen, sulfur, or phosphorous.

The term “hydroxyl group” is represented by the formula —OH.

“Ketogenic composition” as used herein refers to a composition comprising one or more ketogenic compounds.

“Ketogenic compound” refers a compound that is capable of elevating ketone body concentrations in a subject. A ketogenic compound may comprise a ketone body precursor, a ketone ester, a ketone salt, or a combination thereof.

“Ketone” or “ketone body”, as used interchangeably herein, refers to a compound or species which is β-hydroxybutyrate (βHB), acetoacetate, acetone, or a combination thereof. A ketone body may be derived from a ketone body precursor, that is, a compound or species which is a precursor to a ketone body and which may be converted or metabolized to a ketone body in a subject.

“Ketone body ester” or “ketone ester” as used herein, refer to an ester of a ketone body, ketone body precursor, or derivative thereof. Any suitable ketone ester known in the art may be used. For example, the ketone ester may be 1,3 butanediol acetoacetate diester.

“Ketone body salt” or “ketone salt” is a salt of a ketone body, ketone body precursor, or derivative thereof. The ketone salt may be combined with a monovalent cation, divalent cation, or alkaline amino acid. Any suitable ketone salt known in the art may be used. For example, the ketone salt may be a BHB salt. The ketone salt may be a BHB mineral salt. For example, the BHB mineral salt may be potassium βHB, sodium βHB, calcium βHB, magnesium βHB, lithium BHB, or any other feasible non-toxic mineral salts of βHB. The ketone salt may be a BHB organic salt. Organic salts of BHB include salts of organic bases such as arginine βHB, lysine βHB, histidine BHB, ornithine βHB, creatine βHB, agmatine βHB, and citrulline βHB. The ketone salt may be a combination of any of the BHB salts. For example, the ketone salt may be sodium beta-hydroxybutyrate and arginine beta-hydroxybutyrate, or beta-hydroxybutyrate sodium salt and beta-hydroxybutyrate potassium salt.

“Nervous system injury” as used herein refers to any injury to the nervous system. The nervous system injury may be a central nervous system injury. The nervous system injury may be a peripheral nervous system injury.

“Peripheral nervous system injury”, “peripheral nerve injury”, or “PNS injury” as used interchangeably herein refers to any injury to the peripheral nervous system.

A “pharmaceutically acceptable excipient,” “pharmaceutically acceptable diluent,” “pharmaceutically acceptable carrier,”, “carrier”, or “pharmaceutically acceptable adjuvant” as used herein means an excipient, diluent, carrier, and/or adjuvant that are useful in preparing a pharmaceutical composition that are generally safe, non-toxic and neither biologically nor otherwise undesirable, and include an excipient, diluent, carrier, and adjuvant that are acceptable for veterinary use and/or human pharmaceutical use, such as those promulgated by the United States Food and Drug Administration.

The term “sample” as used herein refers to any physical sample that includes a cell or a cell extract from a cell, a tissue, or an organ including a biopsy sample. The sample can be from a biological source such as a subject or animal, or a portion thereof, or can be from a cell culture. Samples from a biological source can be from a normal or an abnormal organism, such as an organism known to be suffering from a condition or a disease state, or any portion thereof. Samples can also be from any fluid, tissue or organ including normal and abnormal (diseased or neoplastic) fluid, tissue or organ. Samples from a subject or animal can be used in the present invention as obtained by the subject or animal and processed or cultured such that cells from the sample can be sustained in vitro as a primary or continuous cell culture or cell line.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc.) and a human). The subject may be a human or a non-human. The subject or patient may be undergoing other forms of treatment.

The term “treatment”, “treat”, “treating” or any grammatical variation thereof as used herein, includes but is not limited to, ameliorating or alleviating a symptom of a disease or condition, reducing, preventing, suppressing, inhibiting, lessening, or affecting the progression and/or severity of an undesired physiological change or a diseased condition. For example, treatment may include preventing neurodegeneration following nervous system injury. Treatment may include enhancing or promoting regeneration of neurons following nervous system injury. Treatment may include enhancing or promoting survival of neurons following nervous system injury. Treatment may include decreasing wound size after nervous system injury. Treatment may include complete elimination of the wound following nervous system injury.

A “therapeutically effective amount,” or “effective dosage” or “effective amount” as used interchangeably herein unless otherwise defined, means a dosage of a drug effective for periods of time necessary, to achieve the desired therapeutic result.

In accordance with the present invention, a suitable single dose size is a dose that is capable of preventing or alleviating a symptom in a subject when administered one or more times over a suitable time period. An effective dosage may be determined by a person skilled in the art and may vary according to factors such as the disease state, age, sex, and weight of the individual, and the ability of the drug to elicit a desired response in the individual. Therapeutically effective amounts for the disclosed compositions can be readily determined by those of ordinary skill in the art.

A therapeutically effective amount may be administered in one or more administrations (e.g., the ketogenic composition may be given as a preventative treatment or therapeutically at any stage of nervous system injury, before or after symptoms, and the like), applications or dosages and is not intended to be limited to a particular formulation, combination or administration route. It is within the scope of the present disclosure that the disclosed ketogenic compositions may be administered at various times during the course of treatment of the subject. The times of administration and dosages used will depend on several factors, such as the disease state, age, sex, and weight of the individual, and the ability of the composition to elicit a desired response in the individual. Administration may be adjusted according to individual need and professional judgment of a person administrating or supervising the administration of the compounds used in the present invention.

“Transesterification” refers to the reaction of an ester with an alcohol to form a new ester compound.

### 2. Ketogenic Compositions

The disclosure provides compositions comprising one or more ketogenic compounds. The ketogenic compound may be any compound capable of elevating ketone body concentrations in a subject. For example, the ketogenic compound may elevate expression of BHB following administration to the subject. The ketogenic compound may be a ketone body precursor, a ketone ester, a ketone salt, or a combination thereof. For example, the ketogenic compound may be a ketone body precursor or derivative thereof. Any suitable ketone body precursor which will be metabolized into a ketone body upon administration to the subject may be used. For example, the ketogenic compound may be 1,3-butanediol, acetoacetate, or BHB moieties or derivatives thereof, including esters and salts thereof. For example, the ketogenic compound may be 1,3-butanediol-acetoacetate diester. The ketogenic compound may be sodium-3-hydroxybutyrate. The ketogenic compound may be R,S-sodium-3-hydroxybutyrate.

The ketogenic compound may be a ketone ester. Any suitable ketone ester may be used in the disclosed ketogenic compositions. Ketone esters may be prepared using any suitable physiologically compatible alcohol. Examples of polyhydric alcohols suitable for preparing such esters include carbohydrates and carbohydrate derivatives, such as carbohydrate alcohols. Examples of carbohydrates include, without limitation, altrose, arabinose, dextrose, erythrose, fructose, galactose, glucose, gulose, idose, lactose, lyxose, mannose, ribose, sucrose, talose, threose, xylose and the like. The ketone ester may be a monoester. The ketone ester may be a diester, The ketone ester may be a polyester. For example, the ketone ester may be 1,3-butanediol-acetoacetate monoester. The ketone ester may be 1,3-butanediol-acetoacetate diester.

The ketogenic compound may be a ketone salt. The ketone salt may be combined with a monovalent cation, divalent cation, or alkaline amino acid. Any suitable ketone salt may be used. For example, the ketone salt may be a BHB salt. The ketone salt may be a BHB mineral salt. For example, the BHB mineral salt may be potassium βHB, sodium βHB, calcium βHB, magnesium βHB, lithium BHB, or any other feasible non-toxic mineral salts of βHB. For example, the ketone salt may be sodium-3-hydroxybutyrate. The ketone salt may be R,S-sodium-3-hydroxybutyrate. The ketone salt may be a BHB organic salt. Organic salts of BHB include salts of organic bases such as arginine βHB, lysine βHB, histidine BHB, ornithine βHB, creatine βHB, agmatine βHB, and citrulline βHB. The ketone salt may be a combination of BHB salts. For example, the ketone salt may be a sodium/potassium BHB mineral salt.

The ketone salt may be mixed into a solution. For example, a βHB mineral salt may be mixed into a solution. The βHB mineral salt may be from 1 to 99% of a solution. For example, the βHB mineral salt may be 5-95%, 10-90%, 20-80%, 30-70%, 40-60%, or about 50% of a solution.

The ketogenic composition may further comprise a pharmaceutically acceptable carrier or excipient. Such carriers may be sterile liquids, such as water and oils. For example, the carrier may be a petroleum oil such as mineral oil; vegetable oil such as peanut oil, soybean oil, or sesame oil; animal oil; or oil of synthetic origin. Suitable carriers also include ethanol, dimethyl sulfoxide, glycerol, silica, alumina, starch, sorbitol, inosital, xylitol, D-xylose, mannitol, powdered cellulose, microcrystalline cellulose, talc, colloidal silicon dioxide, calcium carbonate, magnesium carbonate, calcium phosphate, calcium aluminium silicate, aluminium hydroxide, sodium starch phosphate, lecithin, and equivalent carriers and diluents. Saline solutions and aqueous dextrose and glycerol solutions can also be employed as liquid carriers. Suitable carriers include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The ketogenic composition may contain minor amounts of wetting or emulsifying agents. The ketogenic composition may contain pH buffering agents.

The ketogenic composition may be in a variety of forms. For example, the ketogenic composition may be in solid form, semi-solid form, or a liquid dosage forms. The ketogenic composition may be in the form of tablets, pills, powders, liquid solutions or suspensions, suppositories, and injectable or infusible solutions. The preferred form depends on the intended mode of administration and therapeutic application.

### 3. Methods of Treating Nervous System Injury

The invention discloses a method of treating nervous system injury in a subject. The method of treating nervous system injury in a subject may comprise administering the ketogenic composition to the subject.

The subject may be diagnosed with nervous system injury. The nervous system injury may be a central nervous system injury. CNS injury may be a brain injury. A brain injury may be any injury to the brain. The brain injury may be a traumatic brain injury, caused by an external force to the head. For example, the traumatic brain injury may be a diffuse axonal injury, a concussion, a contusion, a coup-countrecoup injury, a recurrent traumatic brain injury (sometimes referred to as second impact syndrome), an open head injury, a closed head injury, a penetrating injury, Shaken Baby Syndrome, Locked in Syndrome, and the like. The brain injury may be an anoxic brain injury, caused by a complete interruption of the supply of oxygen to the brain. The brain injury may be a hypoxic brain injury, caused by inadequate supply of oxygen to the brain. Examples of anoxic and hypoxic brain injuries include, but are not limited to, hypoxic ischemic encephalopathy, diffuse cerebral hypoxia, focal cerebral ischemia, global cerebral ischemia, and cerebral infarction. CNS injury may be a spinal cord injury (SCI). Spinal cord injury may be any injury to the spinal cord. The SCI may be caused by direct trauma. The SCI may be caused by compression by bone fragments, hematoma, or disc material. The SCI may be at one or more of the cervical vertebrae, thoracic vertebrae, lumbar vertebrae, or sacral vertebrae. The SCI may be to one or more of the cervical cord, thoracic cord, lumbrosacral vertebrae, conus, occiput, or one or more nerves of the cauda equina.

The nervous system injury may be a peripheral nervous system injury. PNS injury may be an injury to any component of the peripheral nervous system. PNS injury may be injury to the sensory-somatic nervous system. PNS injury may be injury to the autonomic nervous system. PNS injury may be injury to one or more cranial nerves. For example, PNS injury may be injury to any one or more of the olfactory nerve, optic nerve, oculomotor nerve, trochlear nerve, trigeminal nerve, abducens nerve, facial nerve, vestibulocochlear nerve, glossopharyngeal nerve, vagus nerve, accessory nerve, or hypoglossal nerve. PNS injury may be injury to any one of more of the spinal nerves. PNS injury may be a traumatic peripheral nerve injury. PNS injury may be a congenital peripheral nerve injury. PNS injury may be an inflammatory peripheral nerve injury. PNS injury may be a toxic peripheral nerve injury. PNS injury may be a tumorous peripheral nerve injury.

The ketogenic composition may be administered to the subject by any suitable route. The ketogenic composition may be administered orally, parentally (including intravenous, subcutaneous, topical, transdermal, intradermal, transmucosal, intraperitoneal, intramuscular, intracapsular, intraorbital, intracardiac, transtracheal, subcutaneous, subcuticular, intraarticular, subcapsular, subarachnoid, intraspinal, and epidural injection) by infusion, by electroporation, or co-administered as a component of any medical device or object to be inserted (temporarily or permanently) into a subject.

The ketogenic composition may be administered in combination with other therapies for the treatment of nervous system injury. Other therapies may include emergency treatments, such as removing clotted blood from the brain, repairing skull fractures, and relieving pressure in the skull. Other therapies may include medications to treat symptoms of the nervous system injury. Other therapies may include medications to reduce other risks associated with nervous system injury. For example, the ketogenic composition may be administered in combination with anti-anxiety medications, anticoagulants, anticonvulsants, antidepressants, diuretics, muscle relaxants, stimulants, and the like. Other therapies may include rehabilitation therapies, including physical therapy, occupational therapy, speech therapy, cognitive therapy, and the like.

Administration of the ketogenic composition may be as a single dose, or multiple doses over a period of time. The ketogenic composition may be administered to the patient at any frequency necessary to achieve the desired therapeutic effect. For example, the ketogenic composition may be administered once to several times every month, every two weeks, every week, or every day. Administration of the ketogenic composition may be repeated until the desired therapeutic effect has been achieved. For example, the ketogenic composition may be administered once to several times over the course of 1 day, 3 days, 5 days, 1 week, 2 weeks, 3 weeks, 1 month, 2 months, 3 months, 4 months, 5 months, 6 months, 7 months, 8 months, 9 months, 10 months, 11 months, 12 months, or more.

The amount of the ketogenic composition to be administered may depend on a variety of factors, such as the route of administration and the seriousness of the condition, and should be decided according to the judgment of the practitioner and each patient's circumstances. The ketogenic composition may be administered in any amount suitable for the treatment of nervous system injury in a subject. An effective amount of the ketogenic composition may cause a partial improvement or a complete elimination of symptoms due to nervous system injury. Treatment may include promoting regeneration of neurons following nervous system injury. Treatment may include enhancing survival of neurons following nervous system injury. Treatment may include decreasing wound size after nervous system injury. Treatment may include complete elimination of the wound following nervous system injury.

Suitable dosage ranges of the ketogenic composition include from about 0.001 mg ketogenic/kg body weight to about 100 mg/kg, about 0.01 mg/kg to about 50 mg/kg, about 0.1 mg/kg to about 25 mg/kg, about 0.5 mg/kg to about 15 mg/kg, about 1 mg/kg to about 10 mg/kg, or about 2.5 mg/kg to about 5 mg/kg. Suitable dosage ranges of insulin include from about 0.001 mg insulin/kg body weight to about 100 mg/kg, about 0.01 mg/kg to about 50 mg/kg, about 0.1 mg/kg to about 25 mg/kg, about 0.5 mg/kg to about 15 mg/kg, about 1 mg/kg to about 10 mg/kg, or about 2.5 mg/kg to about 5 mg/kg.

In addition, in vitro assays may optionally be employed to help identify optimal dosage ranges. Effective doses may be extrapolated from dose-response curves derived from in vitro or animal model test systems.

### 4. EXAMPLES

### Example 1—Administration of a Ketogenic Composition Enhances Neuronal Regeneration Following Injury

Ketone bodies serve as alternative fuel for the brain when glucose availability is limited. In humans, BHB is synthesized in the liver from acetoacetate, the first ketone produced in the fasting state. Its biosynthesis is catalyzed by the enzyme beta-hydroxybutyrate dehydrogenase. The effect of ketone bodies on cell regeneration in primary neuronal cell cultures was tested by using a scratch assay that was followed for 24 hours with a CYTOSMART system.

Cortical neurons from embryonic (E18) Sprague Dawley rats were isolated and cultured as described in Katnic, Guerrero et al. 2006. Briefly, excised brains were digested with 0.25% trypsin. Isolated cells were suspended in DMEM supplemented with fetal bovine serum (FBS, 10%, heat inactivated), penicillin (100 IU/ml), streptomycin (100 mg/ml) and amphotericin B1 (0.25 mg/ml) (Antibiotic/Antimycotic) and plated on poly-L-lysine coated coverslips. Following 24 hour incubation, the DMEM solution was replaced with Neurobasal media supplemented with B-27 (10 ml) and 0.5 mM L-glutamine. 3 week old rat primary neurons were exposed to 2 mM R,S-Sodium-3-hydroxibutyrate. The cell monolayer was scraped with a P100 pipette tip to create a scratch. Cell debris was removed by washing with culture medium. 100 snapshots were taken of the cell migration/cell regeneration into the scratch space over 24 hours by CytoSmart system.

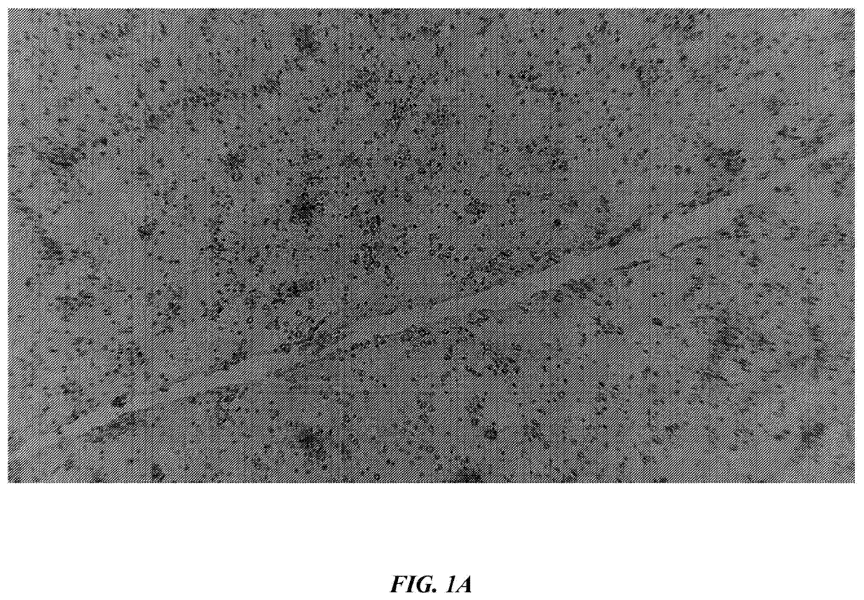

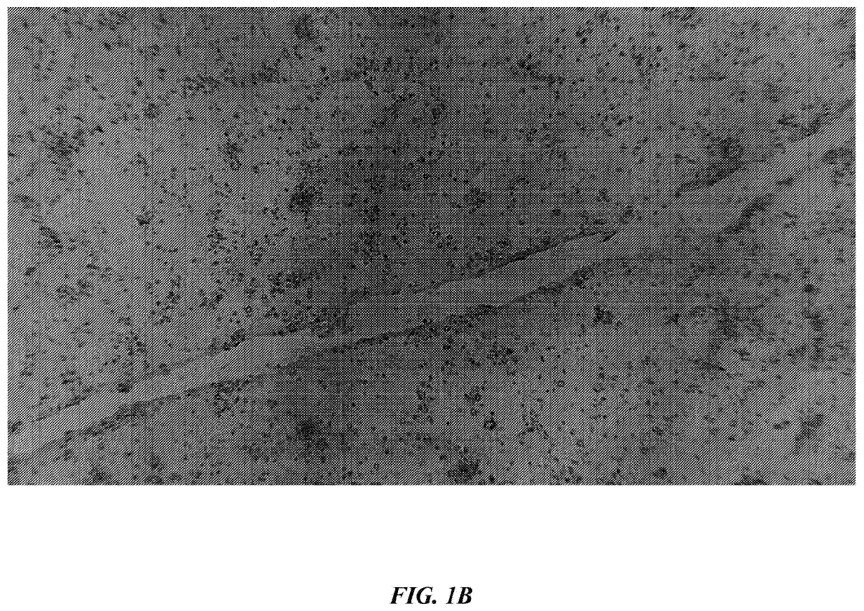

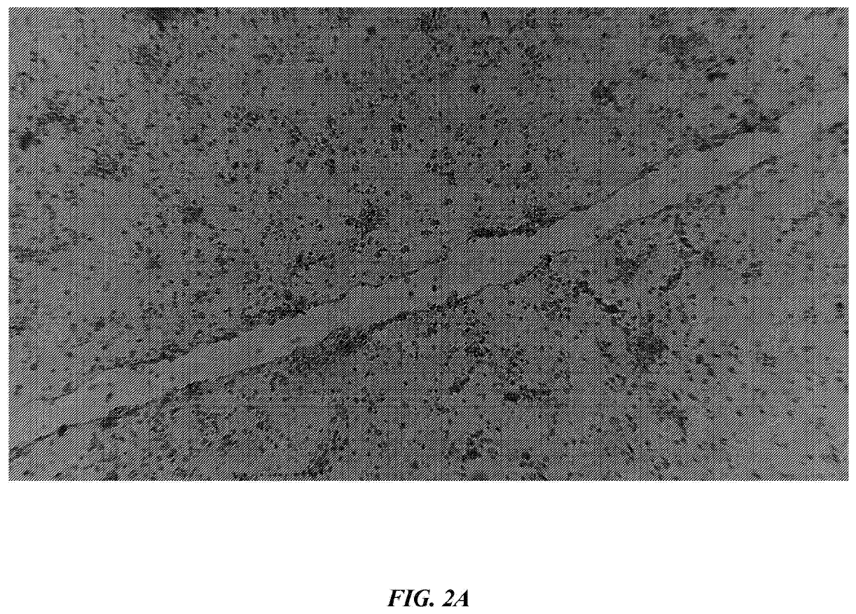

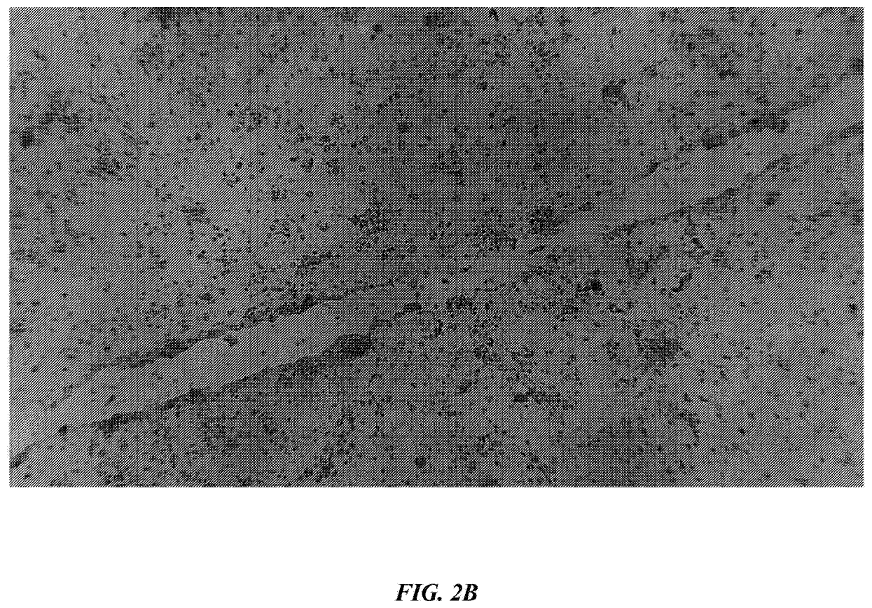

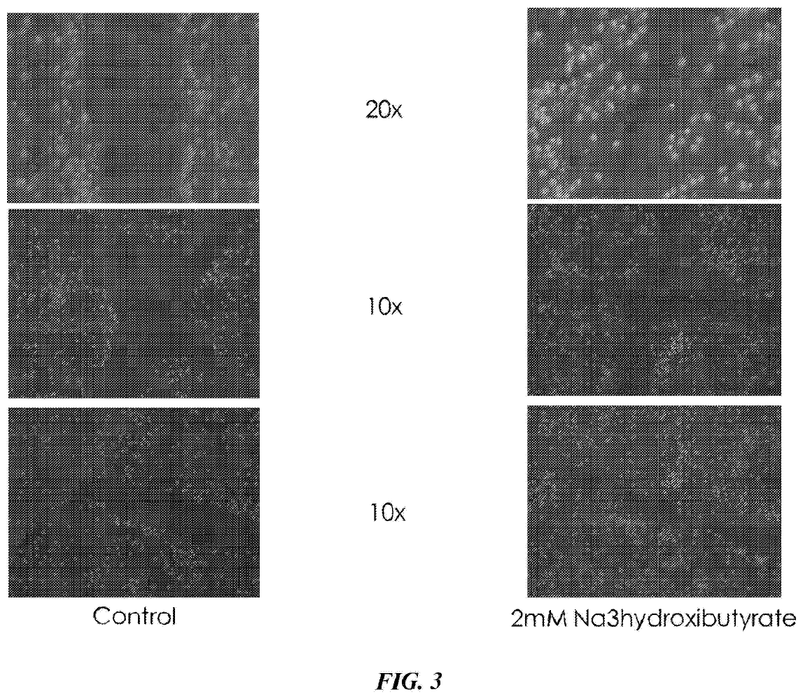

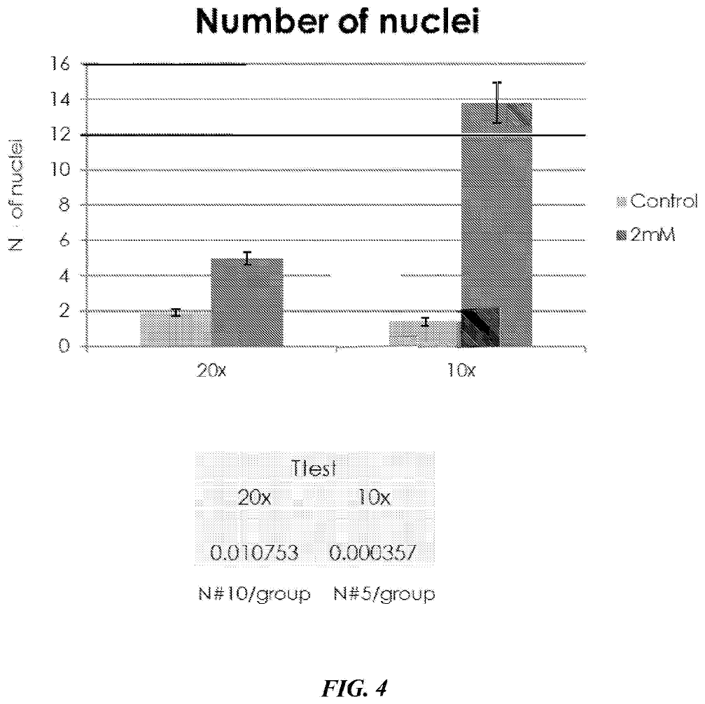

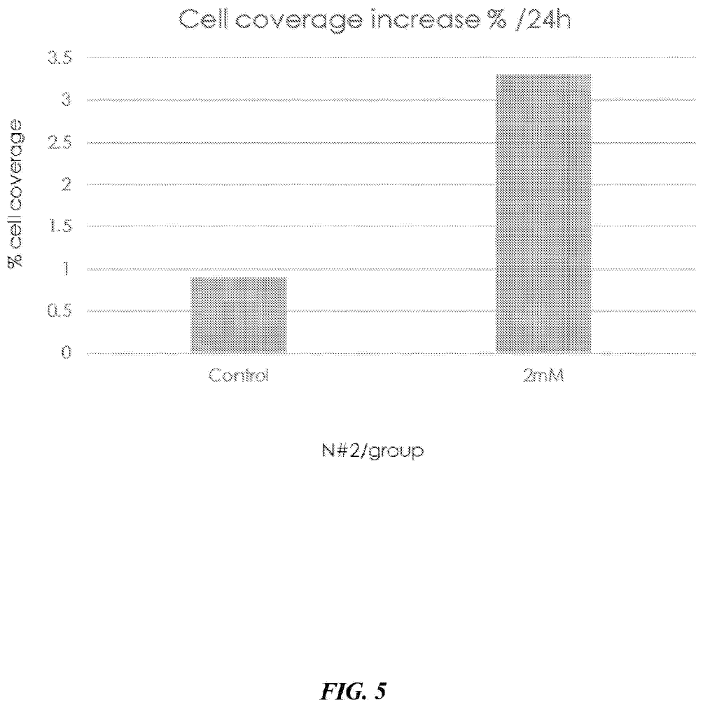

Cell cultures treated with R,S-Sodium-3-hydroxibutyrate showed more intense cell migration and regeneration of the damaged area over the 24-hour period (compare FIGS. 1A-1B versus FIGS. 2A-2B). As shown in FIG. 3 and FIG. 4, significantly more DAPI stained cell nuclei were found in the scratch space area in cell cultures with 2 mM R,S-Sodium-3-hydroxibutyrate at 20× (p=0.01) and at 10× (p=0.0004) magnification photos. The cell coverage increased by about 3.3% with treatment, while it was about 0.9% in the control group. See FIG. 5.

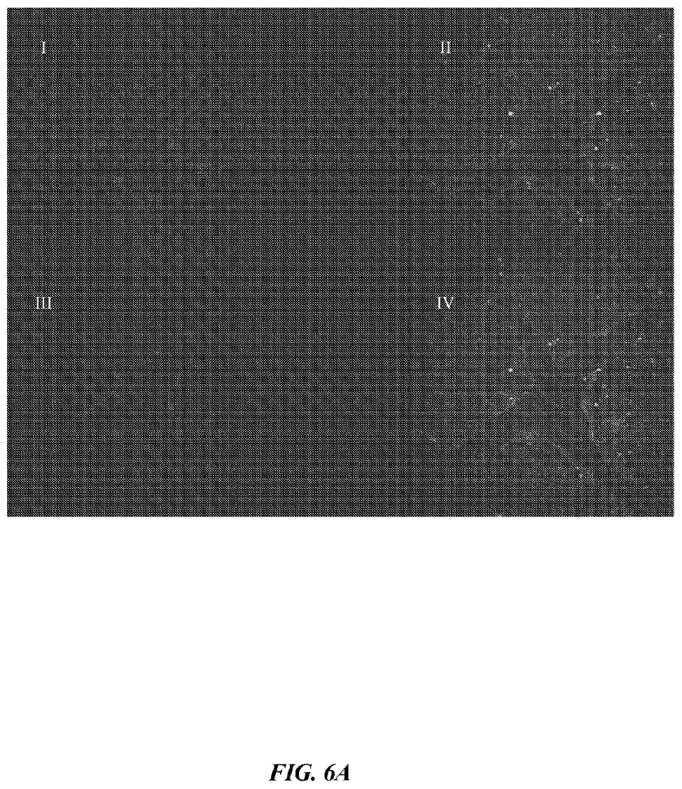

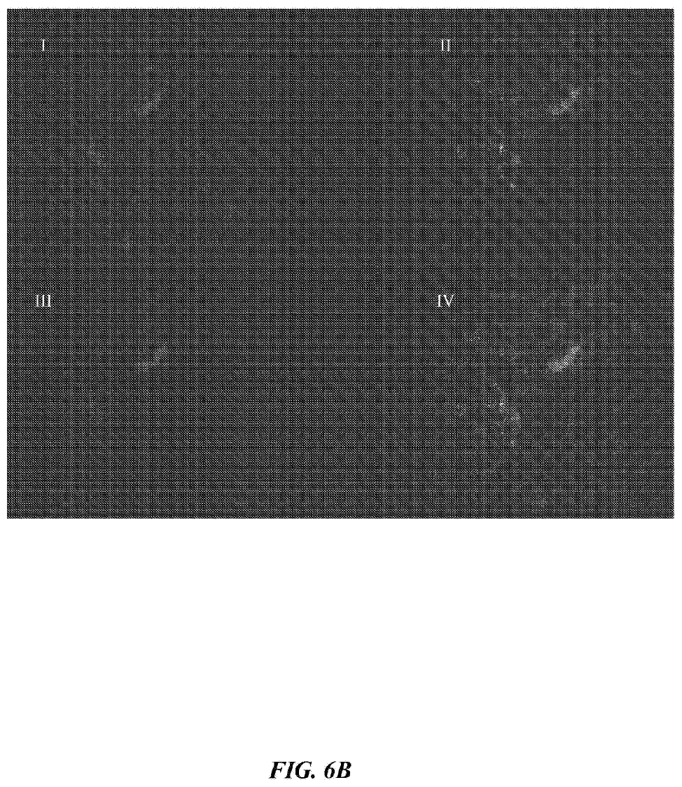

The primary neurons underwent immunofluorescence staining. This fluorescence imaging on the scratch assay (after 24 hours/3-week old primary neurons) can be seen in FIGS. 6A-6B, 7A-7B, 8A-8B, and 9A-9B. The images have been lightened for illustrative purposes. Each of these figures includes four (4) images, labeled as I-IV. In image I, the cell nuclei were stained blue and are depicted; in image II, the Beta tubulin was stained green and is depicted; in image III, the synapsins were stained red and are depicted; and in image IV, the combination of stains of images I-III are depicted. As background, tubulin is a protein present in all cells as a heterodimer of two similar polypeptides α and β, which assemble to form microtubules. Proper organization of microtubules is essential for several cellular functions, such as mitosis, meiosis, some forms of organellar movement, intracellular transport, flagellar movement and cytoskeletal functions. Synapsins are phosphoproteins found in the cytoplasmic surface of synaptic vesicles of the CNS and PNS. Synapsin I is involved in the regulation of axonogenesis and synaptogenesis wherein it serves as a substrate of various protein kinases including those activated by cAMP, calcium/calmodulin, mitogens, and cyclins. As such, it was desired to view both as effected by administration of ketogenic compounds.

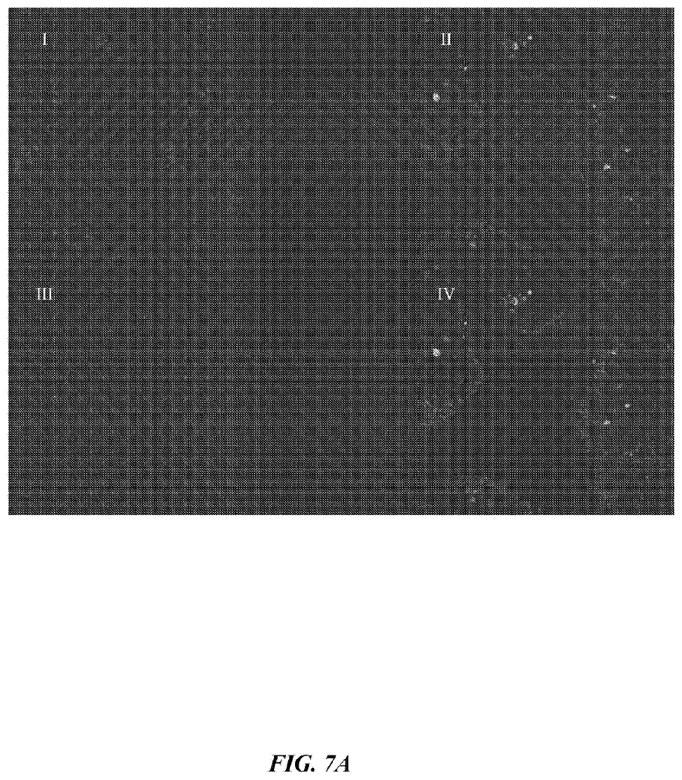

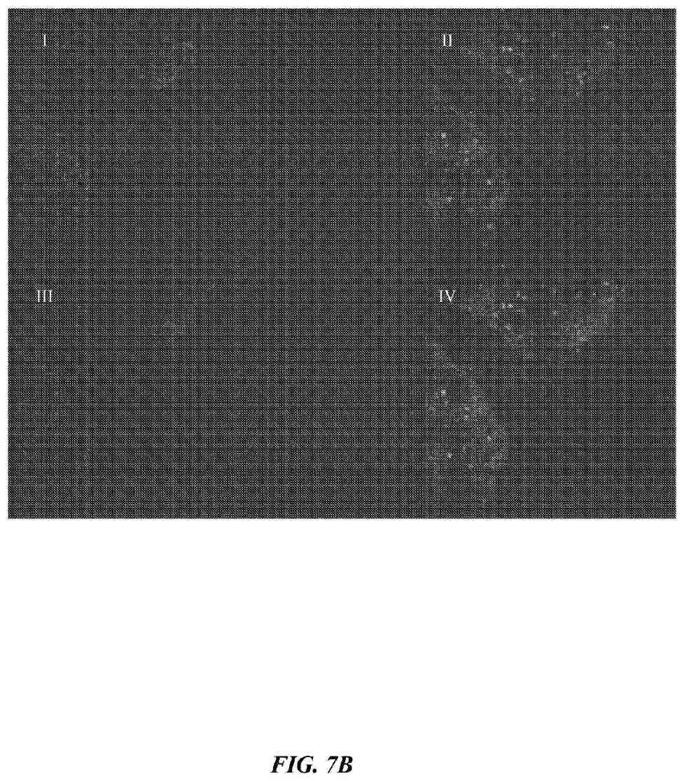

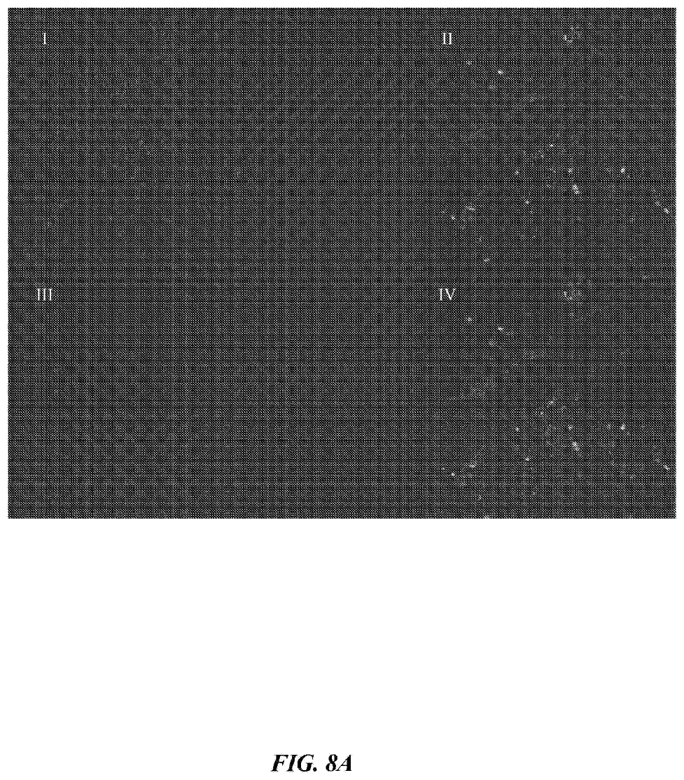

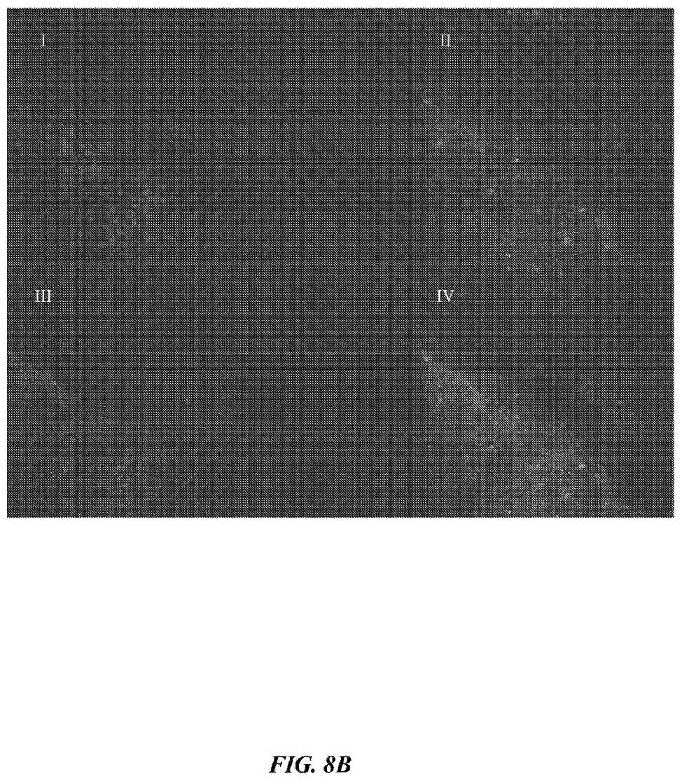

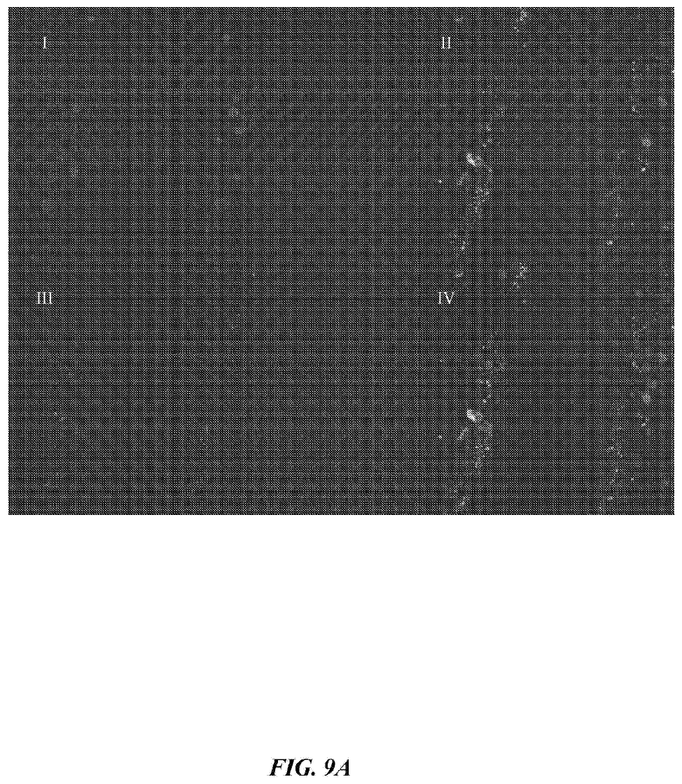

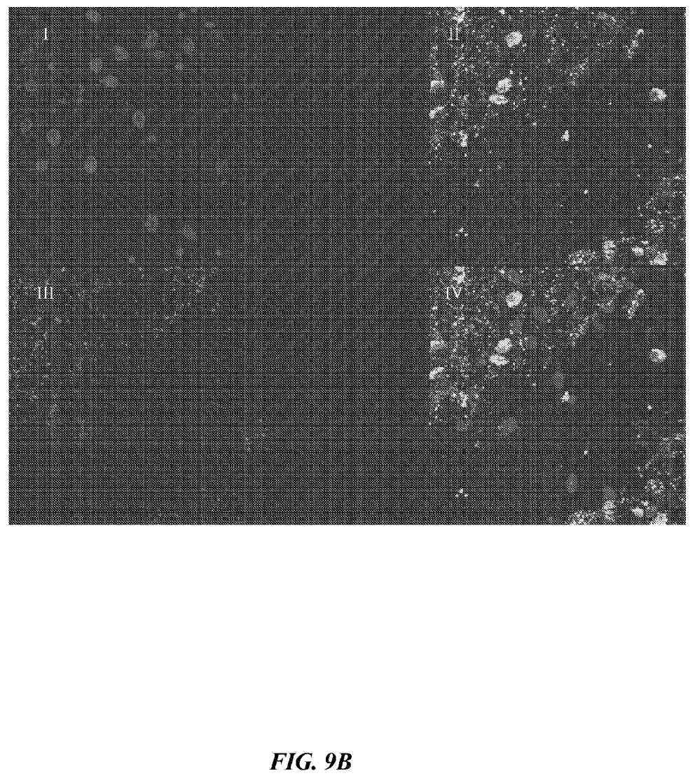

FIGS. 6A-6B and 7A-7B compare the control group to the test group at 10× and 20×, respectively. More cell nuclei are visible around the regeneration site, along with higher density of synapsins and tubulin, as compared to control. FIGS. 8A-8B and 9A-9B compare the control group to the test group at 20× and 60×, respectively. More cell nuclei are visible around the regeneration site and in the scratch area, along with higher density of synapsins and tubulin, as compared to control.

As shown in the above experiments, significantly more cells were found to migrate into the damaged area when the cell culture was treated with R,S-Sodium-3-hydroxibutyrate and the cell coverage was higher than in control cell cultures. Markers that are associated with cytoskeleton and synapsins were denser in treated neuronal culture at the regeneration site as revealed by fluorescence microscopy. These above results illustrate potential applications for ketogenic compounds for use in methods of treating traumatic brain injury or other nervous system injuries.

For reasons of completeness, various aspects of the invention are set out in the following numbered clauses:

Clause 1. A method of treating nervous system injury in a subject, the method comprising administering to the subject a composition comprising one or more ketogenic compounds.

Clause 2. The method of clause 1, wherein the nervous system injury is a central nervous system injury.

Clause 3. The method of clause 2, wherein the central nervous system injury is a brain injury.

Clause 4. The method of clause 2, wherein the central nervous system injury is a spinal cord injury.

Clause 5. The method of clause 1, wherein the nervous system injury is a peripheral nervous system injury.

Clause 6. The method of clause 1, wherein the composition comprises one or more ketogenic compounds selected from the group consisting of a ketone ester, a ketone salt, a ketone body precursor, and a combination thereof.

Clause 7. The method of clause 6, wherein the one or more ketogenic compounds is a ketone ester.

Clause 8. The method of clause 7, wherein the ketone ester is 1,3-butanediol-acetoacetate diester.

Clause 9. The method of clause 6, wherein the one or more ketogenic compounds is a ketone salt.

Clause 10. The method of clause 9, wherein the ketone salt is a (3-hydroxybutyrate salt.

Clause 11. The method of clause 9, wherein the ketone salt is a β-hydroxybutyrate mineral salt.

Clause 12. The method of clause 9, wherein the ketone salt is a sodium-β-hydroxybutyrate salt.

Clause 13. The method of clause 9, wherein the ketone salt is R,S-sodium-3-hydroxybutyrate.

Clause 14. The method of clause 1, wherein the composition enhances neuronal regeneration following nervous system injury.

Clause 15. The method of clause 1, wherein the subject is a vertebrate.

Clause 16. The method of clause 1, wherein the subject is a human.

What is claimed is:

1. A method of treating nervous system injury in a subject, the method comprising administering to the subject a composition comprising one or more ketogenic compounds.

2. The method of claim 1, wherein the nervous system injury is a central nervous system injury.

3. The method of claim 2, wherein the central nervous system injury is a brain injury.

4. The method of claim 2, wherein the central nervous system injury is a spinal cord injury.

5. The method of claim 1, wherein the nervous system injury is a peripheral nervous system injury.

6. The method of claim 1, wherein the composition comprises one or more ketogenic compounds selected from the group consisting of a ketone ester, a ketone salt, a ketone body precursor, and a combination thereof.

7. The method of claim 6, wherein the one or more ketogenic compounds is a ketone ester.

8. The method of claim 7, wherein the ketone ester is 1,3-butanediol-acetoacetate diester.

9. The method of claim 6, wherein the one or more ketogenic compounds is a ketone salt.

10. The method of claim 9, wherein the ketone salt is a β-hydroxybutyrate salt.

11. The method of claim 9, wherein the ketone salt is a β-hydroxybutyrate mineral salt.

12. The method of claim 9, wherein the ketone salt is a sodium-β-hydroxybutyrate salt.

13. The method of claim 9, wherein the ketone salt is R,S-sodium-3-hydroxybutyrate.

14. The method of claim 1, wherein the composition enhances neuronal regeneration following nervous system injury.

15. The method of claim 1, wherein the subject is a vertebrate.

16. The method of claim 1, wherein the subject is a human.
